(19) United States
(12) Patent Application Publication
(10) Pub. No.: US 2025/0217730 A1
(43) Pub. Date: Jul. 3, 2025

(54) SYSTEMS AND METHODS FOR PERSONALIZED RANKED ALERTS

(71) Applicant: KONINKLIJKE PHILIPS N.V., EINDHOVEN (NL)

(72) Inventors: Tiblets Zeray DEMEWEZ, EINDHOVEN (NL); Qi GAO, EINDHOVEN (NL); Mauro BARBIERI, EINDHOVEN (NL); Johannes Henricus Maria KORST, EINDHOVEN (NL); Severius Petrus Paulus PRONK, VUGHT (NL)

(21) Appl. No.: 18/848,604

(22) PCT Filed: Mar. 20, 2023
(86) PCT No.: PCT/EP2023/057029

§ 371 Date: Sep. 19, 2024

Related U.S. Application Data
(60) Provisional application No. 63/323,689, filed on Mar. 25, 2022.

(51) Int. Cl. G06Q 10/0631; G05B 23/02; G06Q 10/0639; G16H 40/40

(52) U.S. Cl. CPC G06Q 10/063112; G05B 23/024; G05B 23/0283; G06Q 10/06398; G16H 40/40

(57) ABSTRACT

In an alerting system, one or more predictive models are trained to generate maintenance alerts for medical devices of a fleet of medical devices based on machine log data received from the medical devices. Historical maintenance alerts data are stored including at least historical maintenance alerts generated by the one or more predictive models for the fleet of medical devices. Instructions are readable and executable by at least one electronic processor to: train an alert ranking machine learning (ML) model to rank alerts of a queue of alerts using the historical maintenance alerts data; receive unresolved alerts for medical devices of the fleet from the one or more predictive models; generate a ranked list of the unresolved alerts allocated to a service engineer (SE) using the trained ranking ML model; and provide, on a display device accessible by the SE, the ranked list of the unresolved alerts allocated to the SE.

### FIELD

The following relates generally to the medical device maintenance arts, medical imaging device maintenance arts, medical device maintenance visualization arts, and related arts.

### BACKGROUND

Maintenance of medical imaging systems and other medical devices such as patient monitoring systems consists of multiple types of maintenance activities. In planned maintenance activities, a field service engineer (FSE) visits the hospital to oil, clean, calibrate, etc. the system at regular intervals (e.g., once, or twice every year, or with a frequency that is determined by the usage of the system, or dynamically scheduled based on remotely monitoring the condition of the system). In addition, there are corrective maintenance activities, that are initiated as a reaction to an issue reported by the hospital. If the issue is severe, then this may result in unplanned down time of the system. The system may not be in operation until the issue is fixed again, either remotely by a remote service engineer (RSE) or on site by an FSE. Unplanned down time can lead to considerable costs for the hospital, as no examinations can be scheduled for some time. It can also lead to patient dissatisfaction, as examinations may have to be rescheduled to a later time.

In addition to the above-mentioned maintenance activities, predictive maintenance activities are used to avoid unplanned downtime. For various parts of a medical imaging system, predictive models have been developed that aim to predict when a part is likely to fail soon, so that the part can be replaced preventively before it fails. These predictive models may be constructed by using a machine learning algorithm that, based on a training set of historical cases, builds a predictive model. For a given subsystem/part p of a given system s, such a predictive model will estimate, for a given a given time window [t,t+w], the probability Pr(p,s,t) that p will fail in this time window. These estimated probabilities can next be used to determine whether it makes sense to preventively replace p in the coming week or weeks. The predictive models analyze log event data that the medical imaging system s produces. Log event data may contain sensor measurements as well as log events in the form of low-level error and warning messages.

Once a predictive model has been tested to perform at a sufficient performance level (considering the probability and cost of false positives as well as false negatives), it can be deployed to monitor many medical imaging systems in the field. The model can be run on recent log event data of each of the systems at regular intervals, e.g., once every hour or day, or it can be triggered dynamically by the availability of new data. If for a system s, it concludes that Pr(p,s,t) exceeds a certain threshold, it can raise an alert. Alternative strategies, such as logged value exceeding a threshold at least k times in 1 successive time units can also be used to raise an alert.

Specialized remote service engineers (RSEs) are trained to review the raised alerts, for example via a workstation computer that shows a ranked list of recent alerts. To each alert of type a priority P(a) is associated, so that all alerts that are raised by the multiple predictive models are simply ordered in order of priority. The RSEs typically consider the alerts in a top-down fashion, addressing the highest priority alerts first. Note that the type a of an alert is based on the predictive model, and likewise the priority P(a) is based on the parameters of the predictive model, such as accuracy, false positive rate (FPR), and estimated repair timeframe window size w.

As the number of alerts increase, identifying high priority alerts that RSEs are well equipped to handle becomes a challenge. Some of the alerts are not interesting to the RSEs or they require some knowledge to resolve the issue. As a result, an important alert may be out of sight and RSEs fail to act on time. Consequently, customers may report an issue when it could have been proactively solved if the RSEs would have time and required skills to resolve the issues. Moreover, alerts that are not reviewed and for which no follow up steps have not been taken (e.g., creating a service case) within a certain time window are removed from the RMW.

The following discloses certain improvements to overcome these problems and others.

### SUMMARY

In one aspect, a non-transitory computer readable medium stores one or more predictive models trained to generate maintenance alerts for medical devices of a fleet of medical devices based on machine log data received from the medical devices, historical maintenance alerts data including at least historical maintenance alerts generated by the one or more predictive models for the fleet of medical devices, and instructions readable and executable by at least one electronic processor to: train an alert ranking machine learning (ML) model to rank alerts of a queue of alerts using the historical maintenance alerts data; receive unresolved alerts for medical devices of the fleet from the one or more predictive models; generate a ranked list of the unresolved alerts allocated to a service engineer (SE) using the trained ranking ML model; and provide, on a display device accessible by the SE, the ranked list of the unresolved alerts allocated to the SE.

In another aspect, a non-transitory computer readable medium stores one or more predictive models trained generate maintenance alerts for medical devices of a fleet of medical devices based on machine log data received from the medical devices, historical maintenance alerts data including at least historical maintenance alerts generated by the one or more predictive models for the fleet of medical devices; and instructions readable and executable by at least one electronic processor to: train an alert ranking machine learning (ML) model to rank alerts of a queue of alerts using the historical maintenance alerts data; receive unresolved alerts for medical devices of the fleet from the one or more predictive models; generate a global ranking the unresolved alerts using the trained ranking ML model; allocate the unresolved alerts amongst a plurality of service engineers (SEs); order the unresolved alerts allocated to the SE in accordance with the global ranking of the unresolved alerts to generate a ranked list of the unresolved alerts allocated to an SE; and provide, on a display device accessible by an SE, the ranked list of the unresolved alerts allocated to that SE.

In another aspect, a non-transitory computer readable medium stores one or more predictive models trained generate maintenance alerts for medical devices of a fleet of medical devices based on machine log data received from the medical devices, historical maintenance alerts data including at least historical maintenance alerts generated by the one or more predictive models for the fleet of medical devices, and instructions readable and executable by at least one electronic processor to: train an alert ranking machine learning (ML) model to rank alerts of a queue of alerts using the historical maintenance alerts data; receive unresolved alerts for medical devices of the fleet from the one or more predictive models; allocate the unresolved alerts amongst a plurality of SEs including the SE; rank the unresolved alerts allocated to the SE using the trained ranking ML model; and provide, on a display device accessible by a service engineer (SE), the ranked list of the unresolved alerts allocated to that SE.

One advantage resides in providing personalized alerts to RSEs for unresolved alerts.

Another advantage resides in providing a personalized list of alerts to corresponding RSEs to improve alert handling time and improve RSE engagement.

Another advantage resides in reduced downtown of medical devices.

Another advantage resides in providing personalized alerts to RSEs for unresolved alerts based on historical alert data.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative system for servicing medical devices in accordance with the present disclosure.

FIGS. 2-4 shows exemplary flow chart operations of the system of FIG. 1.

### DETAILED DESCRIPTION

A disadvantage of existing workflows for handling maintenance alerts is that the distribution and ordering of alerts are not generally personalized. Similar alerts are presented to all RSEs. This leaves room for improvement. By presenting a personalized list of alerts to each of the RSEs as disclosed herein, the alert handling time and the RSE engagement improves. As a result, the resolution time of customers' issues improves.

The following relates to prioritizing or ranking alerts for individual remote service engineers (RSEs). In daily operation, machine logs received from imaging devices of a fleet of imaging devices are analyzed by diagnostic models and the model outputs scored to generate alerts relating to preventative maintenance tasks that should be performed. The alerts are allocated to RSEs on staff, and are presented to the respective RSEs via a user interface such as a workstation computer.

Disclosed herein are approaches for ranking the alerts on an individualized basis using information on the alerts obtained from a case management database (referred to herein as “alert characteristics”), such as the predictive model that generated the alert, deadlines of the alerts, customer contract terms, customer satisfaction information (if available; e.g. a customer with low satisfaction may be ranked higher), the number of similar systems that the customer has (e.g., if the customer has several similar systems then downtime for the system subject to the alert may be less critical), and modalities or system types for which an RSE has expertise, RSE overall experience, training, or the like. Notably, these latter alert characteristics are RSE specific. Probabilities (or other metrics) for ranking the alerts are computed for (alert, RSE) pairs based on the alert characteristics, and for a given RSE the alerts are ranked based on the computed probabilities.

In some embodiments, probabilities for the alerts are computed and then the alerts are allocated to RSEs and displayed ranked based on the probabilities. In other embodiments, the alerts are allocated to RSEs and then, on a per-RSE basis, the probabilities are computed, and the alerts allocated to that RSE are ranked. This latter approach can improve computational efficiency as only one probability need be computed for each alert, whereas the first embodiment requires computing for each alert the probabilities for all RSEs. On the other hand, the first embodiment provides the probabilities (or other metrics) prior to allocating the alerts to the RSEs, e.g. the probabilities can be computed for all (alert, RSE) pairs, and hence in the first embodiment the probabilities can be used to determine the allocation, e.g. by allocating alerts with high probabilities for one particular RSE to that RSE.

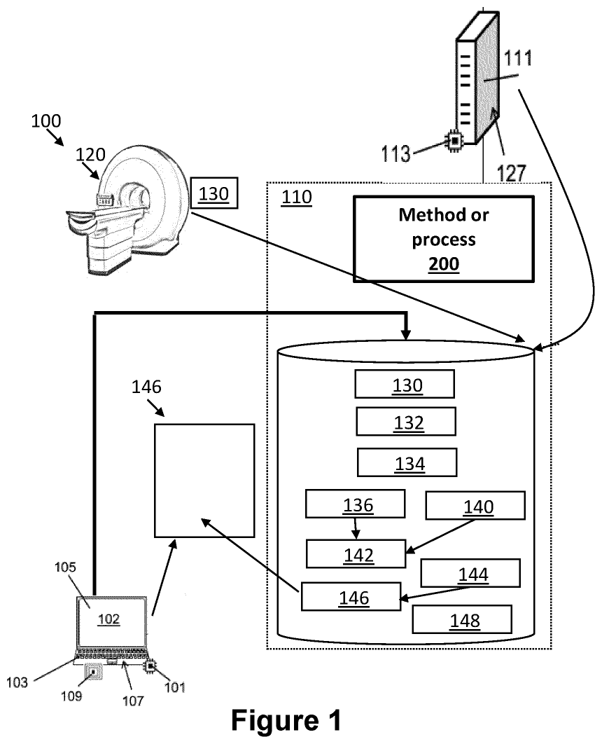

With reference to FIG. 1, an illustrative servicing support system 100 for supporting a service engineer in servicing an electronic device 120 (e.g., a medical imaging device—also referred to as a medical device, an imaging device, imaging scanner, and variants thereof) is diagrammatically shown. By way of some non-limiting illustrative examples, the medical imaging device under service may be a magnetic resonance imaging (MRI) scanner, a computed tomography (CT) scanner, a positron emission tomography (PET) scanner, a gamma camera for performing single photon emission computed tomography (SPECT), an interventional radiology (IR) device, or so forth. (More generally, the disclosed approach can be applied in conjunction with any type of computerized device that automatically generates log data, e.g., the approach could be applied to a commercial airliner, radiation therapy device, or so forth).

As shown in FIG. 1, the servicing support system 100 includes, or is accessible by, a service device 102 that may for example be a workstation or electronic processing device used by a user (e.g., a service engineer (SE), such as a remote SE (RSE)). The service device 102 may for example be a workstation computer accessed by an RSE. The service device 102 can be a desktop computer or a personal device, such as a mobile computer system such as a laptop or smart device. While a single workstation 102 for a single RSE is shown in FIG. 1 by way of illustration, more generally each RSE working at any given time will be assigned to a corresponding workstation 102. For example, if six RSEs are working at a given time, each will typically work at a corresponding workstation 102 so that there will be six workstations 102 active at that time.

The service device 102 includes a display device 105 via which alerts generated by predictive failure models are displayed, optionally along with likely root cause and service action recommendation information if this is provided by the predictive model. The service device 102 also preferably allows the service engineer to interact with the servicing support system via at least one user input device 103 such a mouse, keyboard, or touchscreen. The service device further includes an electronic processer 101 and non-transitory storage medium 107 (internal components which are diagrammatically indicated in FIG. 1). The non-transitory storage medium 107 stores instructions which are readable and executable by the electronic processor 101 for interfacing with the servicing support system 100. The service device 102 also includes a communication interface 109 to communicate with a backend server or processing device 111, which typically implements the computational aspects of the servicing support system 100 (e.g., the server 111 has the processing power for implementing computationally complex aspects of the servicing support system 100). Such communication interfaces 109 include, for example, a wired and/or wireless Ethernet interface (e.g., in the case in which the service device 102 is an RSE workstation); or in the case in which the service device 102 is a portable FSE device the interface 109 may be a wireless Wi-Fi or 4G/5G interface or the like for connection to the Internet and/or an intranet. Some aspects of the servicing support system 100 may also be implemented by cloud processing or other remote processing (that is, the server computer 111 may be embodied as a cloud-based computing resource comprising a plurality of interconnected servers).

In illustrative FIG. 1, the servicing support system further includes a backend 110 (e.g., implemented and/or owned by the imaging device vendor or leased by the vendor from a cloud computing service provider). The backend 110 receives log data (e.g., a machine log automatically generated by the medical imaging device 120, a service log for the medical imaging device 120, and/or so forth) on a continuous or occasional basis (e.g., in some setups the imaging device 120 uploads machine log entries to the backend 110 on a daily basis). The backend processing for performing predictive fault modeling using predictive models. The backend server 111 is equipped with an electronic processor 113 (diagrammatically indicated internal component). The server 111 is equipped with non-transitory storage medium 127 (internal components which are diagrammatically indicated in FIG. 1). While a single server computer is shown, it will be appreciated that the backend 110 may more generally be implemented on a single server computer, or a server cluster, or a cloud computing resource comprising ad hoc-interconnected server computers, or so forth. Furthermore, while FIG. 1 shows a single medical imaging device 120, more generally the database backend 110 will receive log data from many medical imaging devices (e.g., tens, hundreds, or more imaging devices) and performs the disclosed processing for a medical imaging device undergoing servicing using the log data generated by that device.

The non-transitory computer readable medium 127 stores machine log data 130 received from the medical device 120. The non-transitory computer readable medium 127 stores one or more predictive models 132 trained generate maintenance alerts for the medical device 120 as part of a fleet of medical devices based on the machine log data 130 received from the medical device(s) 120. The non-transitory computer readable medium 127 also stores historical maintenance alerts data including at least historical maintenance alerts 134 generated by the one or more predictive models 132 for the fleet of medical devices 120. In some examples, the historical maintenance alerts data further includes information on the predictive models 132 that generated the respective historical maintenance alerts, deadlines of the respective historical maintenance alerts, customer contract terms associated with the medical devices of the respective historical maintenance, and customer satisfaction information associated with the medical devices of the respective historical maintenance.

The non-transitory storage medium 127 also stores instructions executable by the electronic processor 113 of the backend server 111 to perform a method 200 of ranking and allocating the maintenance alerts generated by the predictive models 132 to RSEs (or, equivalently, to their corresponding workstations 102 to which the respective RSEs are logged into).

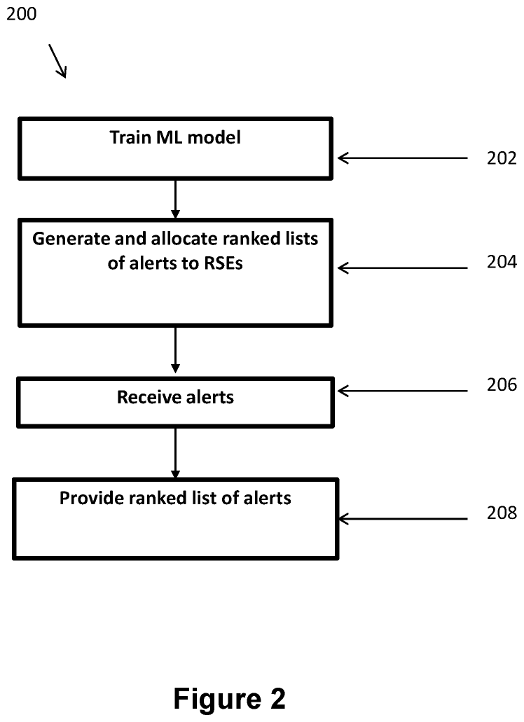

With continuing reference to FIG. 1 and further reference to FIGS. 2-4, an illustrative embodiment of the method 200 executable by the electronic processor 113 of the backend server 111 is diagrammatically shown as a flowchart. In some examples, the method 200 may be performed at least in part by cloud processing.

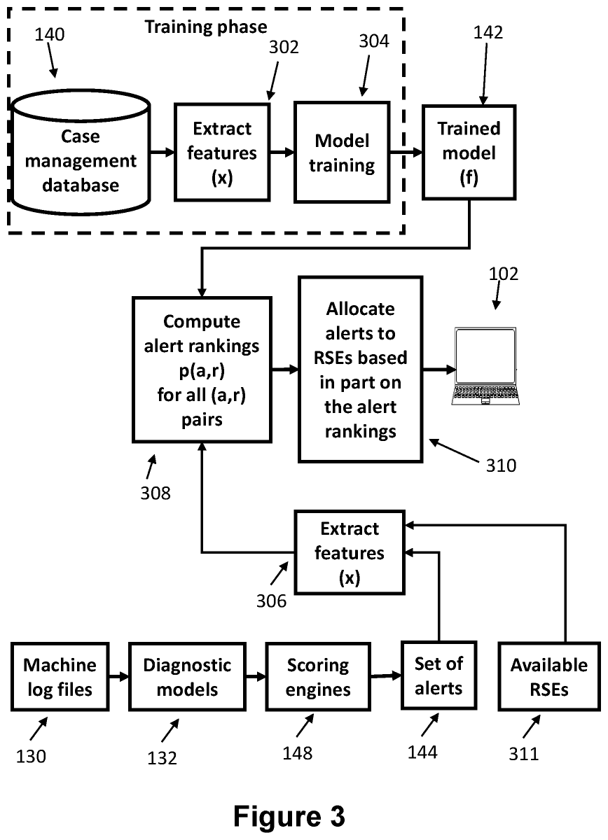

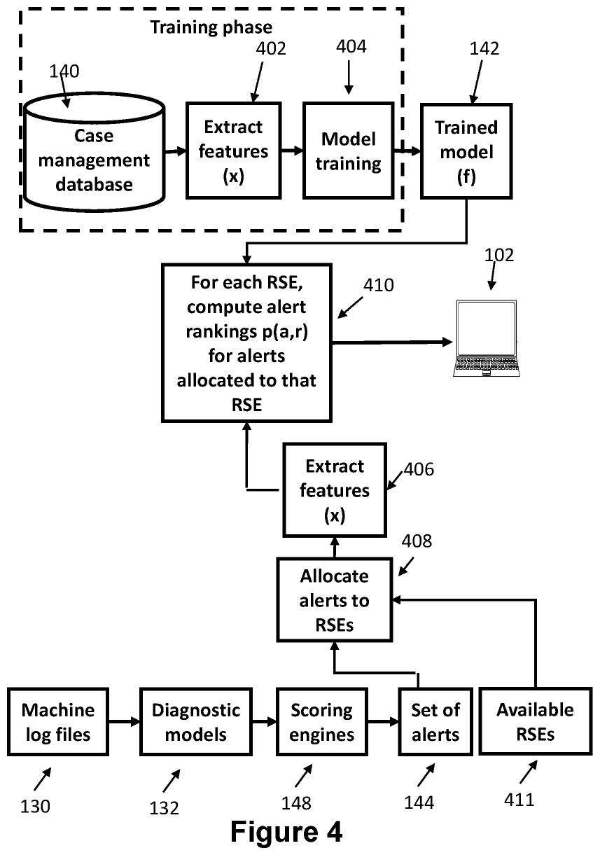

At an operation 202, an alert ranking machine learning (ML) model 136 is trained to rank alerts 138 of a queue of alerts using the historical maintenance alerts data. To do so, as shown in FIGS. 3 and 4, the historical maintenance alerts data is retrieved from a case management database 140 stored in the non-transitory computer readable medium 127, and features are extracted from the retrieved data to train the ML model 136 (as shown in operations 302, 402 and 304, 404) to generate a trained model 142.

At an operation 204, unresolved alerts 144 for medical devices of the fleet are received from the predictive model(s) 134.

At an operation 206, ranked lists 146 of the unresolved alerts 144 are generated and allocated to SEs using the trained ranking ML model 142. To do so, the unresolved alerts 144 are allocated amongst a plurality of SEs, and the unresolved alerts 144 allocated to each SE are ranked using the trained ML model 142.

At an operation 208, the ranked list 146 of the unresolved alerts 144 allocated to each SE are shown on the display device 105 of the service device 102 accessible by the corresponding SE. Each SE receives the ranked list 146 as the unresolved alerts 144 allocated to that particular SE.

In one embodiment, the alerts 144 are ranked based on expertise data including modalities or system types of the one or more medical devices 120 for which each SE has expertise. To do so, alert-SE pairs are generated back on the expertise data. To generate the pairs, probabilities for each alert-SE pair are computed based on the historical alert and the expertise data, and the alerts 144 are allocated to corresponding SEs based on the computed probabilities (for example, for display on a corresponding service device 102 operable by each SE).

This embodiment is shown in more detail in FIG. 3. To generate the alert-SE pairs, the machine log data or files 130 are input to the predictive model(s) 134. A scoring engine 148 implemented in the backend server 111 is configured to score the predictive model(s) 132 to generate the alert-SE pairs. The unresolved alerts 144 are received an analyzed to extract features therefrom (shown at 306). The ranking for each alert-SE pair are then computed for each pair (shown at 308), and allocated to the available SEs 311 based at least on the rankings (shown at 310). The ranking of an alert a for an SE r is also denoted herein as p(a,r). Note that if the ranking is computed based on SE-specific attributes (e.g., SE expertise, training, or so forth) then the ranking of the same alert a₁ for different SEs r₁ and r₂ may be different, e.g. p(a₁,r₁)≠p(a₁,r₂). In the embodiment of FIG. 3 the allocation 310 to available RSEs 311 is performed after the ranking 308 of the alerts, and so the rankings p(a,r) can advantageously be used as a factor in deciding the allocations. For example, alerts that are well suited for a particular RSE (e.g., having high p(a,r) for that RSE) can be allocated to that RSE. On the other hand, this approach requires that the ranking of an alert be computed for every RSE (since it is not known at ranking step 308 which RSE a given alert will be assigned). Hence, Nₐ*Nᵣ probabilities p(a,r) are computed, where Nₐ is the number of alerts and Nᵣ is the number of RSEs to whom the alerts are to be allocated.

Another embodiment of the ranking operation 206 is shown in FIG. 4. The alert-SE pairs are generated as in FIG. 3 (with the extracting operation labeled as 406 in lieu of 306 as in FIG. 3). The unresolved alerts 144 are first allocated to corresponding available SEs 411 (shown at 408), and then the alerts are ranked for each SE (shown at 410). In this embodiment the ranking 410 is performed after the allocation 408, so that the probabilities p(a,r) are unavailable for use in determining the allocation. However, in this embodiment the number of probabilities that are calculated is lower, namely Nₐ, since for each alert the probability p(a,r) need only be calculated for the single RSE to whom that alert a is allocated.

In some embodiments, the historical maintenance alerts data further includes performance data of the plurality of SEs in resolving the historical maintenance alerts 134. The alert ranking ML model 142 is trained to rank the alerts 144 of the queue of alerts using the historical maintenance alerts data 134 including the performance data of the plurality of SEs, and the ranking of the unresolved alerts 144 allocated to the SE using the trained ranking ML model 142 is based in part on the performance data of the SE.

In other embodiments, the generation of the ranked list 146 of the unresolved alerts 144 allocated to the SE includes generating a global ranking the unresolved alerts 144 using the trained ranking ML model 142. The unresolved alerts 144 are allocated amongst a plurality of SEs, and the unresolved alerts 144 allocated to the SE are ordered in accordance with the global ranking of the unresolved alerts 144.

### Examples

The following describes the system 100 and the method 200 in more detail. The system 100 is configured to present a personalized ranking of alerts generated by diagnostic models tailored to individual RSEs based on their history and skills. The alert handling history and profile of RSEs together with alert characteristics are used as input to an algorithm to estimate the probability of an alert being reviewed by an RSE. Subsequently, the alerts with their corresponding probability estimates are partitioned by RSE. The probability estimates are then ordered in descending order to provide personalized alerts to each of the RSEs that will later be presented in RMW.

A personalized ranking engine can be embedded into an end-to-end proactive monitoring process, and takes as input alerts generated by diagnostic models, alert handling history of each RSE, the profile of each RSE, alert characteristics, and so forth.

Alerts generated by diagnostic models using scoring engine and historical data are provided to the ranking engine where alerts are partitioned and ordered. The RMW takes the output of the ranking engine, which is basically set of ordered alerts per RSE, and presents it on RMW. Assuming that an alert will appear in the ranking of only one RSE, one could think of optimizing some objective function that considers for each (alert, RSE) pair (a, e) the probability that alert a is solved successfully by engineer e as well as the time that e requires to solve a.

To avoid multiple RSEs simultaneously selecting the same alert, a single alert is assigned to one RSE. Alternatively, alerts can be moved from the queue of one RSE to the queue of another one. Assuming round-the-clock service, RSEs will start and stop working overtime. As such the alerts in the queue of an RSE that stops working are redirected to the queue of other RSEs. Preferably this does not require additional time to handle the alerts. Additionally, if an RSE starts his/her working shift, then the alerts for which he/she is specifically well-skilled to solve are redirected to his or her queue.

The ranking engine is built using an algorithm that takes a set of alerts that are generated by diagnostic models. In addition, the RSEs profile and their corresponding alert handling history as well as alert characteristics are provided as an input to the engine. Some of the alert characteristics are, for example, the number of successfully resolved alerts, the average alert resolution time, the proportion of resolved alerts per modality, the number of resolved alerts of a similar part or subsystem, the success factor of previous handled alerts posts, the similarity of the new alert compared to the previously resolved alerts, and so forth.

To create the ranking engine, the algorithm is trained using historical data. For that, data preparation is required to convert the input data into features the algorithm can take as an input. Moreover, experimenting with different techniques is required to select the right approach.

To describe the approach mathematically, let A={a₁, a₂, . . . , aₙ} be a set of alerts, R={r₁, r₂, . . . , rₘ} be the available RSEs and C={c₁, c₂, . . . , cₖ} be the alert characteristics. Every alert a∈A has k characteristics. Suppose a∈A be an alert with deadline dₐ and p(a, r) be the probability that alert a is reviewed by RSE r∈R at dₐ. p(a,r) can be calculated as p(a,r)=ƒ(x(a, r, 1), x(a, r, 2), . . . , x(a, r, k)) where x(a, r, i) represents the features that are derived from alert characteristics and historical alert resolution of RSEs. After calculating the probability for each RSE and partitioning over the RSE, the probability is sorted in descending order and presented in RMW.

The steps taken to produce the ranked alerts are as follows: (1) gather historical data of the RSEs. The data reflects the whole experience of the RSE in handling alerts; (2) fetch alerts generated by diagnostic models that are due to be published in RMW. These alerts have an alert creation day and a deadline; (3) get the values of x(a,r,i) (features) for each alert a and RSE r and alert characteristics I; (4) calculate p(a,r) for all alerts and RSEs; and sort p(a,r) in descending order for each RSE r.

Whenever the RSE opens RMW, the ranking engine is triggered to create list of alerts to publish in real time. The engine fetches historical data and alert characteristics, it then converts the data to features. Afterwards, for each alert the engine calculates the probability an alert is reviewed by an RSE. These probabilities are partitioned by RSEs, sorted in descending order, and then published in RMW.

At the start of each day, the newly arrived alerts and the alerts that are already in the queues of the RSEs are taken as a single set that must be redistributed and ranked over the available RSEs, considering which RSEs will be available in the next time period. In that way, one could also dynamically determine the probability that an alert will be handled in the coming period by a given RSE depending on the queue of alerts that will be presented to the RSE in the next period. This could be estimated by assuming that the RSE would handle the alerts in order of the proposed ranking and by using the time distribution that the given RSE would need to solve the alerts that precede the given alert in the given ranking.

An additional embodiment is to show the personalized ranked and selected alerts directly to staff at hospitals, e.g., the biomedical engineers (as known as biomeds). The biomeds in hospitals are responsible for maximizing the efficiency of the systems at the facility to deliver the best level of patient care. In some cases, they are responsible for specific maintenance activities of the medical imaging systems. Selected alerts are ranked based on their expertise (often biomeds have limited knowledge or are less experienced than the service engineers of OEMs) and service contract that the hospital has.

A non-transitory storage medium includes any medium for storing or transmitting information in a form readable by a machine (e.g., a computer). For instance, a machine-readable medium includes read only memory (“ROM”), solid state drive (SSD), flash memory, or other electronic storage medium; a hard disk drive, RAID array, or other magnetic disk storage media; an optical disk or other optical storage media; or so forth.

The methods illustrated throughout the specification, may be implemented as instructions stored on a non-transitory storage medium and read and executed by a computer or other electronic processor.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing:
one or more predictive models trained to generate maintenance alerts for medical devices of a fleet of medical devices based on machine log data received from the medical devices;
historical maintenance alerts data including at least historical maintenance alerts generated by the one or more predictive models for the fleet of medical devices;
instructions readable and executable by at least one electronic processor to:
train an alert ranking machine learning (ML) model to rank alerts of a queue of alerts using the historical maintenance alerts data;
receive unresolved alerts for medical devices of the fleet from the one or more predictive models;
generate a ranked list of the unresolved alerts allocated to a service engineer (SE) using the trained ranking ML model; and
provide, on a display device accessible by the SE, the ranked list of the unresolved alerts allocated to the SE.

2. The non-transitory computer readable medium of claim 1, wherein the generation of the ranked list of the unresolved alerts allocated to the SE includes:
allocating the unresolved alerts amongst a plurality of SEs including the SE; and
ranking the unresolved alerts allocated to the SE using the trained ranking ML model.

3. The non-transitory computer readable medium of claim 2 wherein:
the historical maintenance alerts data further includes performance data of the plurality of SEs in resolving the historical maintenance alerts, and
the alert ranking ML model is trained to rank the alerts of the queue of alerts using the historical maintenance alerts data including the performance data of the plurality of SEs, and
the ranking of the unresolved alerts allocated to the SE using the trained ranking ML model is based in part on the performance data of the SE.

4. The non-transitory computer readable medium of claim 1, wherein the generation of the ranked list of the unresolved alerts allocated to the SE includes:
generating a global ranking the unresolved alerts using the trained ranking ML model;
allocating the unresolved alerts amongst a plurality of SEs including the SE; and
ordering the unresolved alerts allocated to the SE in accordance with the global ranking of the unresolved alerts.

5. The non-transitory computer readable medium of claim 1, wherein the historical maintenance alerts data further includes information on the predictive models that generated the respective historical maintenance alerts, deadlines of the respective historical maintenance alerts, customer contract terms associated with the medical devices of the respective historical maintenance, and customer satisfaction information associated with the medical devices of the respective historical maintenance.

6. The non-transitory computer readable medium of claim 1, wherein the alerts are ranked based on expertise data including modalities or system types of the one or more medical devices for which each SE has expertise.

7. The non-transitory computer readable medium of claim 6, wherein the instructions further include:
generating alert-SE pairs based on the expertise data.

8. The non-transitory computer readable medium of claim 7, wherein the instructions further include:
computing probabilities for each alert-SE pair based on the historical alert data and the expertise data; and
allocating the alerts to corresponding SEs based on the computed probabilities.

9. The non-transitory computer readable medium of claim 8, wherein the alerts allocated to the corresponding SEs are displayed on a corresponding display device operable by each SE.

10. The non-transitory computer readable medium of claim 7, wherein the instructions further include:
allocating the alerts to corresponding SEs; and
computing probabilities for each alert allocated to each corresponding SE based on the historical alert data, the performance data, and the expertise data.

11. The non-transitory computer readable medium of claim 10, wherein the alerts allocated to the corresponding SEs are displayed as a ranked list of alerts on a corresponding display device operable by each SE.

12. The non-transitory computer readable medium of claim 1, wherein the one or more medical devices comprise medical imaging devices.

13. A non-transitory computer readable medium storing:
one or more predictive models trained generate maintenance alerts for medical devices of a fleet of medical devices based on machine log data received from the medical devices;
historical maintenance alerts data including at least historical maintenance alerts generated by the one or more predictive models for the fleet of medical devices; and
instructions readable and executable by at least one electronic processor to:
train an alert ranking machine learning (ML) model to rank alerts of a queue of alerts using the historical maintenance alerts data;
receive unresolved alerts for medical devices of the fleet from the one or more predictive models;
generate a global ranking the unresolved alerts using the trained ranking ML model;
allocate the unresolved alerts amongst a plurality of service engineers (SEs);
order the unresolved alerts allocated to the SE in accordance with the global ranking of the unresolved alerts to generate a ranked list of the unresolved alerts allocated to an SE; and
provide, on a display device accessible by an SE, the ranked list of the unresolved alerts allocated to that SE.

14. The non-transitory computer readable medium of claim 13, wherein the historical maintenance alerts data further includes information on the predictive models that generated the respective historical maintenance alerts, deadlines of the respective historical maintenance alerts, customer contract terms associated with the medical devices of the respective historical maintenance, and customer satisfaction information associated with the medical devices of the respective historical maintenance.

15. The non-transitory computer readable medium of claim 13, wherein the alerts are ranked based on expertise data including modalities or system types of the one or more medical devices for which each SE has expertise.

16. The non-transitory computer readable medium of claim 15, wherein the instructions further include:
generating alert-SE pairs based on the expertise data.

17. The non-transitory computer readable medium of claim 16, wherein the instructions further include:
compute probabilities for each alert-SE pair based on the historical alert data and the expertise data; and
allocate the alerts to corresponding SEs based on the computed probabilities.

18. The non-transitory computer readable medium of claim 17, wherein the alerts allocated to the corresponding SEs are displayed on a corresponding display device operable by each SE.

19. A non-transitory computer readable medium storing:
one or more predictive models trained generate maintenance alerts for medical devices of a fleet of medical devices based on machine log data received from the medical devices;
historical maintenance alerts data including at least historical maintenance alerts generated by the one or more predictive models for the fleet of medical devices; and
instructions readable and executable by at least one electronic processor to:
train an alert ranking machine learning (ML) model to rank alerts of a queue of alerts using the historical maintenance alerts data;
receive unresolved alerts for medical devices of the fleet from the one or more predictive models;
allocate the unresolved alerts amongst a plurality of SEs including the SE;
rank the unresolved alerts allocated to the SE using the trained ranking ML model; and
provide, on a display device accessible by a service engineer (SE,) the ranked list of the unresolved alerts allocated to that SE.

20. The non-transitory computer readable medium of claim 19, wherein:
the historical maintenance alerts data further includes performance data of the plurality of SEs in resolving the historical maintenance alerts, and
the alert ranking ML model is trained to rank the alerts of the queue of alerts using the historical maintenance alerts data including the performance data of the plurality of SEs, and
the ranking of the unresolved alerts allocated to the SE using the trained ranking ML model is based in part on the performance data of the SE.
